(19) United States
(12) Patent Application Publication
(10) Pub. No.: US 2003/0225333 A1
(43) Pub. Date: Dec. 4, 2003

(54) Indicia bearing and color-coded medical device and associated methods

(76) Inventors: Mark William Roby, Lake Mary, FL (US); Saundra Lynn Roby, Lake Mary, FL (US)

Correspondence Address: Att: Jeffrey S. Whittle, Bracewell & Patterson, L.L.P., P.O. Box 61389, Houston, TX, 77208-1389, US

(21) Appl. No.: 10/190,025

(22) Filed: Jul. 3, 2002

Related U.S. Application Data
(63) Continuation-in-part of application No. 10/156,452, filed on May 28, 2002.

(51) Int. Cl. A61B 5/02

(52) U.S. Cl. 600/490

(57) ABSTRACT

A blood pressure monitor for sports enthusiasts is provided which preferably includes a cuff having a fabric portion and an inflatable bladder portion and adapted to be positioned to substantially surround a portion of one of the patient's arms, an inflator in fluid communication with the bladder portion of the cuff to inflate the bladder portion responsive to the user, a measurer in fluid communication with the bladder portion of the cuff for measuring blood pressure of the patient when the bladder portion substantially surrounds the portion of the patient's arm, a first tube having a first end portion connected to and in fluid communication with the cuff and a second end portion connected to and in fluid communication with the inflator, and a second tube having a first end portion connected to and in fluid communication with the cuff and a second end portion connected to and in fluid communication with the measurer. Each of the first and second tubes further preferably is visibly colored with at least one of a first color associated with a sports team to thereby define a first sports team color and a second contrasting color also associated with the sports team to thereby define a second sports team color.

### CROSS-REFERENCE TO RELATED APPLICATIONS

This is a continuation-in-part (CIP) of U.S. patent application Ser. No. 10/156,452, filed on May 28, 2002 and titled “Indicia Bearing and Color-Coded Medical Device and Associated Methods”, which is incorporated herein by reference in its entirety.

### FIELD OF THE INVENTION

The present invention relates to the field of medical devices and, more particularly, to blood pressure monitors for sports enthusiasts and methods for retrofitting existing blood pressure monitors for sports blood pressure monitors as well as methods of doing business related to such blood pressure monitors.

### BACKGROUND OF THE INVENTION

Blood pressure is a measure of the force of blood inside an artery. The measurement is taken by momentarily stopping the flow of blood in an artery (usually by inflating a cuff around the upper arm) and then listening for the sound of the blood beginning to flow through the artery again as air is released from the cuff. As blood flows through the artery, it can be heard through a stethoscope placed on the skin over the artery inside the arm. Blood pressure is recorded as two measurements: (1) the reading on a measuring gauge when blood flow is first heard is called the systolic pressure. Systolic pressure represents the peak blood pressure that occurs when heart contracts; (2) the reading on a measuring gauge when blood flow can no longer be heard is the diastolic pressure. Diastolic pressure represents the lowest blood pressure that occurs when the heart relaxes between beats. These two pressures are often expressed in millimeters of mercury (mm Hg) on a measuring gauge.

Two general types of blood pressure monitors are commonly available, i.e., manual and automatic. Manual devices usually include an arm cuff, a squeeze bulb for inflation, a stethoscope or microphone, and a mechanical gauge or column of mercury to measure the blood pressure. Electronic monitors are battery-operated monitors which use a microphone to detect blood pulsing in the artery instead of having to listen with a stethoscope. The cuff, which is attached to a patient's wrist or upper arm, is connected to an electronic monitor that automatically inflates and deflates the cuff when the start button is pressed. In consequence, a reading displays on the monitor, which records a patient's pulse as well as blood pressure.

One of the common and practical problems with conventional medical devices including blood pressure monitors is patient apprehension of the instrument before and during its application to the patient's body parts.

### SUMMARY OF THE INVENTION

With the foregoing in mind, the present invention advantageously provides a plurality of different types of blood pressure monitors that are specifically designed for sports enthusiasts. The present invention also advantageously provides medical personnel a momento of a school sports team they attended or of which they are a fan, of a sports team for whom they are a fan, and as a reminder or symbol of collegiate memories, loyalties, or friendships. Further, the present invention provides an advertisement vehicle for a sports team. Still further, the present invention advantageously provides sports enthusiasts or others methods to easily retrofit existing medical devices with sports indicia to indicate endorsements, loyalties, or the like.

Solutions to a problem such as patient apprehension include applying indicia that is appealing to a patient to a medical device to help create a comfortable setting for the patient. It is believed that providing appealing indicia, such as illustrations or graphical designs, on an outer surface of the medical device may serve to at least partially pacify such an apprehensive patient prior to applying that surface to the patient's body.

According to an embodiment of the present invention, a blood pressure monitor is provided for sports enthusiasts for monitoring blood pressure of a patient. The monitor preferably has a cuff having a fabric portion and an inflatable bladder portion and adapted to be positioned to substantially surround a portion of one of the patient's arms. The monitor also preferably has a bulb in fluid communication with the bladder portion of the cuff to inflate the bladder portion responsive to squeeze pressure from a user, measuring means in fluid communication with the bladder portion for measuring blood pressure of the patient when the bladder portion substantially surrounds the patient's arm, a first tube having a first end portion connected to and in fluid communication with the cuff and a second end portion connected to and in fluid communication with the bulb, and a second tube having a first end portion connected to and in fluid communication with the cuff and a second end portion connected to and in fluid communication with the measuring means. The fabric portion of the cuff advantageously has sports indicia associated with a sports team positioned thereon, which are readily visible and recognizable to the user and the patient. The first and second tubes are advantageously colored with at least one of a first color associated with the sports team to thereby define a first sports team color and a second contrasting color also associated with the sports team to thereby define a second sports team color.

Preferably, the sports team may include at least one of the following: a school sports team, a professional sports team and a club sports team. To further target sports teams, the fabric portion of the cuff and the bulb can be colored with at least one of the first and second sports team colors. Additionally, the bulb can further have the same sports indicia. In the present invention, for example, the sports indicia have colors corresponding to the first and second sports team colors and may include sports logos, sponsor logos, licensed logos, character's logos, and other famous indicia readily recognizable by both the user and the patient. Representative examples of the sports indicia include college, high school, graduate school, professional, club, civic or community, church and other sports teams' logos.

The measuring means, for example, can be provided by a dial having a needle moving responsive to pressure in the cuff. The face of the dial has the same sports indicia positioned thereon. Alternatively, for example, the measuring means can be provided by a casing including a surface thereof having the sports indicia positioned thereon, a tubular housing connected to the casing and having mercury material positioned therein, and measurement indicators associated with the tubular housing to indicate blood pressure measurements responsive to the position of the mercury material positioned within the tubular housing. The mercury material preferably is responsive to pressure in the cuff. To further target sports teams, the tubular housing can be colored with the first sports team color, and the surface of the casing can be colored with the second sports team color. Additionally, a lower end portion of the tubular housing can have a shape substantially similar to a ball associated with the sports team, e.g. basketball, soccer ball, football, volleyball, or baseball.

According to another embodiment of the present invention, a blood pressure monitor is provided for sports enthusiasts for monitoring blood pressure of a patient. The blood pressure monitor preferably has a cuff having a fabric portion and an inflatable bladder portion and adapted to be positioned to substantially surround a portion of one of the patient's arms, a bulb in fluid communication with the bladder portion of the cuff to inflate the bladder portion responsive to squeeze pressure from the user, measuring means in fluid communication with the bladder portion for measuring blood pressure of the patient when the bladder portion substantially surrounds the patient's arm, a first tube having a first end portion connected to and in fluid communication with the cuff and a second end portion connected to and in fluid communication with the bulb, and a second tube having a first end portion connected to and in fluid communication with the cuff and a second end portion connected to and in fluid communication with the measuring means. The first and second tubes are advantageously colored with at least one of a first color associated with a sports team to thereby define a first sports team color and a second contrasting color also associated with the sports team to thereby define a second sports team color. The fabric portion of the cuff and the bulb, for example, can be colored with at least one of the first and second sports team colors. More preferably, the fabric portion of the cuff and the bulb can have sports indicia associated with the same sports team positioned thereon.

According to still another embodiment of the present invention, a blood pressure monitor is advantageously provided for sports enthusiasts for monitoring blood pressure of a patient. The blood pressure monitor preferably has a cuff having a fabric portion and an inflatable bladder portion and adapted to be positioned to substantially surround a portion of one of the patient's arms, a gauge which is in fluid communication with the bladder portion of the cuff to inflate and deflate the bladder portion responsive to a user as well as to measure the patient's blood pressure, and first and second tubes each having a first end portion connected to and in fluid communication with the cuff and a second end portion connected to and in fluid communication with the gauge. The fabric portion of the cuff advantageously has sports indicia associated with a sports team positioned thereon, which are readily visible and recognizable to the user and the patient. The first and second tubes are also advantageously colored with at least one of a first color associated with the sports team to thereby define a first sports team color and a second contrasting color also associated with the sports team to thereby define a second sports team color.

According to yet another embodiment of the present invention, a blood pressure monitor is advantageously provided for sports enthusiasts for monitoring blood pressure of a patient. The blood pressure monitor preferably has a cuff having a fabric portion and an inflatable bladder portion and adapted to be positioned to substantially surround a portion of one of the patient's arms, a gauge which is in fluid communication with the bladder portion of the cuff to inflate and deflate the bladder portion responsive to a user as well as to measure the patient's blood pressure, and first and second tubes each having a first end portion connected to and in fluid communication with the cuff and a second end portion connected to and in fluid communication with the gauge. The first and second tubes are advantageously colored with at least one of a first color associated with the sports team to thereby define a first sports team color and a second contrasting color also associated with the sports team to thereby define a second sports team color. The fabric portion of the cuff, for example, can be colored with at least one of the first and second sports team colors and further have sports indicia positioned thereon. Additionally, the gauge has a surface advantageously colored with at least one of the first and second sports team colors and can further have the sports indicia positioned thereon.

The present invention also advantageously provides a method of retrofitting an existing blood pressure monitor for a sports blood pressure monitor. The method preferably includes replacing first and second tubes of the existing blood pressure monitor with third and fourth tubes being visibly colored with at least one of a first color associated with a sports team to thereby define a first sports team color and a second contrasting color also associated with the sports team to thereby define a second sports team color.

A retrofitting method of the present invention, for example, can further include replacing a cuff of the existing blood pressure monitor with a different cuff which is preferably colored with at least one of the first and second sports team colors. More preferably, the cuff can have sports indicia associated with the sports team imprinted thereon.

Alternatively, the retrofitting method can further include replacing a bulb of the existing blood pressure monitor with a different bulb which is preferably colored with at least one of the first and second sports team colors. More preferably, the bulb can have sports indicia associated with the sports team imprinted thereon.

The present invention further advantageously provides a method of doing business which preferably includes obtaining a license from at least one sports teams to use sports team identifiers associated with the sports team in association with a blood pressure monitor, positioning sports team identifiers on a blood pressure monitor so that a potential user of the blood pressure monitor readily recognizes that the blood pressure monitor is associated with the sports team, selling the blood pressure monitor to the potential user, and distributing the blood pressure monitor to the potential user after purchase of the blood pressure monitor. The method can further include advertising the blood pressure monitor associated with the sports team.

Preferably, the sports team can include at least one of the following: a school sports team, a professional sports team and a club sports team. Still preferably, the sports identifier is positioned on at least one of the following: a fabric portion of a cuff, a bulb, a surface of measuring means of the blood pressure monitor.

Another method of doing business according to the present invention preferably includes obtaining a license from each of a plurality of different sports teams to use sports team identifiers associated with each of the plurality of sports teams in association with a plurality of blood pressure monitors and manufacturing a plurality of blood pressure monitors. Each of the plurality of blood pressure monitors preferably has a different sports team identifier associated therewith. Such method can further include advertising each of the plurality of blood pressure monitors associated with each of the plurality of sports teams or selling the plurality of blood pressure monitors to the potential user and distributing the plurality of blood pressure monitors to the potential user after purchase of the blood pressure monitors.

Therefore, a blood pressure monitor for sports enthusiasts and methods of doing business disclosed herein advantageously create a sports medical product or device for sports enthusiasts as well as pacifies an apprehensive patient by assisting medical personnel in focusing the patient on the device rather than the procedure or check up being performed. Additionally, the present invention advantageously provides memorabilia and/or gift ideas for sports enthusiasts and helps build team loyalty.

### BRIEF DESCRIPTION OF THE DRAWING

Some of the features, advantages, and benefits of the present invention having been stated, others will become apparent as the description proceeds when taken in conjunction with the accompanying drawings in which:

FIG. 1 is a perspective view of a blood pressure monitor according to an embodiment of the present invention;

FIG. 2A is a perspective view of a blood pressure monitor according to another embodiment of the present invention;

FIG. 2B is an enlarged fragmentary perspective view of a lower end portion of a tubular housing and casing of a blood pressure monitor shown in FIG. 2A according to the present invention; and

FIG. 3 is a perspective view of a blood pressure monitor according to yet another embodiment of the present invention.

### DETAILED DESCRIPTION

The present invention will now be described more fully hereinafter with reference to the accompanying drawings which illustrated preferred embodiments of the invention. This invention may, however, be embodied in many different forms and should not be construed as limited to the illustrated embodiments set forth herein. Rather, these illustrated embodiments are provided so that this disclosure will be thorough and complete, and will fully convey the scope of the invention to those skilled in the art. Like numbers refer to like elements throughout, and prime and/or double prime notation are used to indicate similar elements in alternative embodiments.

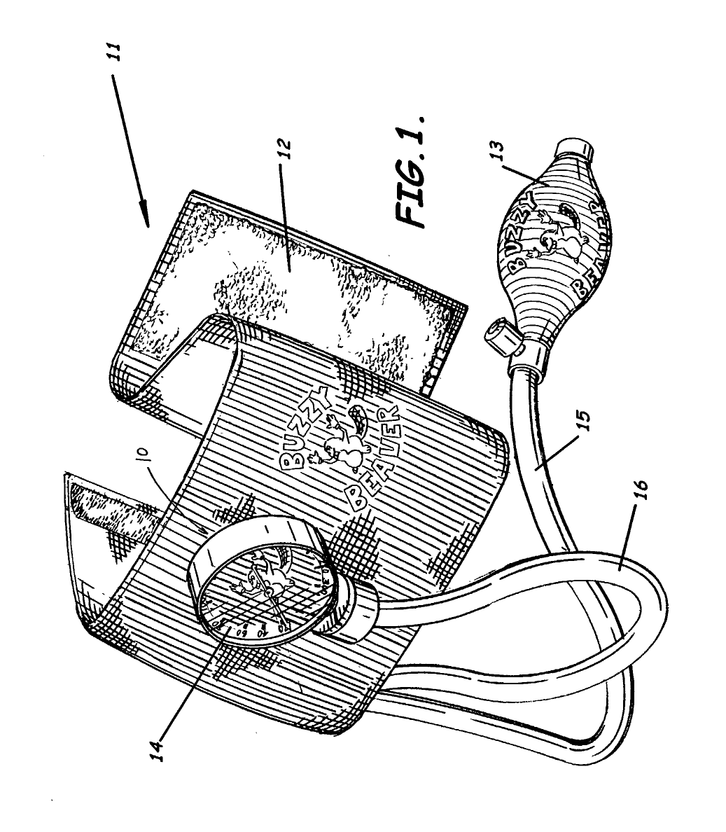

FIG. 1 illustrates a blood pressure monitor 11 for sports enthusiasts according to an embodiment of the present invention. The blood pressure monitor 11 preferably has a cuff 12 adapted to be positioned to substantially surround a patient's arm. The cuff 12 preferably has a fabric portion and an inflatable bladder portion associated with the fabric portion. The blood pressure monitor 12 also preferably has a bulb 13 which is in fluid communication with the inflatable bladder portion of the cuff 12 to inflate the bladder portion in response to squeeze pressure from a user, measuring means 10 which is also in fluid communication with the bladder portion of the cuff 12 for measuring blood pressure of the patient when the cuff 12 substantially surrounds the patient's arm, a first tube 15 which is connected to and in fluid communication with the cuff 12 at one end portion and connected to and in fluid communication with the bulb 13 at the other end portion, and a second tube 16 which is connected to and in fluid communication with the cuff 12 at one end portion and connected to and in fluid communication with the measuring means 10 at the other end portion.

The measuring means 10, for example, can be provided by a dial 14 which preferably has a needle moving clockwise and counterclockwise in response to the pressure in the cuff 12. In actual use, the cuff 12 is wrapped around a patient's arm. Air is pumped into the bladder portion of the cuff 12 by means of the squeeze bulb 13. The bladder portion of the cuff 12 is then inflated to momentarily stop the flow of blood in an artery and subsequently deflated as air is released from the cuff 12. In consequence, the needle in the dial 14 moves responsive to the pressure in the cuff 12, thus enabling the measurement of the patient's blood pressure.

Each of the first tube 15 and the second tube 16 is preferably colored with at least one of a first color associated with a sports team to thereby define a first sports team color and a second contrasting color also associated with the sports team to thereby define a second sports team color. The fabric portion of the cuff 12, the surface of the bulb 13, and/or the face of the dial 14 are preferably colored with at least one of the first and second sports team colors. To further target the sports team, the fabric portion of the cuff 12, the surface of the bulb 13 and/or the face of the dial 14 can preferably have sports indicia associated with the sports team positioned thereon. The sports indicia preferably have colors corresponding to the first and second sports team colors and can include sports logos, sponsor logos, licensed logos, character's logos, and other famous indicia that are readily recognizable by both the user and the patient. Representative examples of the sports indicia include college, high school, graduate school, professional, club, civic or community, church and other sports teams' logos, mascots, names, slogans, abbreviations, initials or marks. As illustrated in FIG. 1, the blood pressure monitor 11 has the Buzzy Beaver logo imprinted on the fabric portion of the cuff 12, the surface of the bulb 13 and the face of the dial 14. Examples of various types of colors or logos and combinations thereof can also be seen in U.S. patent application Ser. No. 10/156,452, filed on May 28, 2002 and titled “Indicia Bearing and Color-Coded Medical Device and Associated Methods”, which is incorporated herein by reference in its entirety.

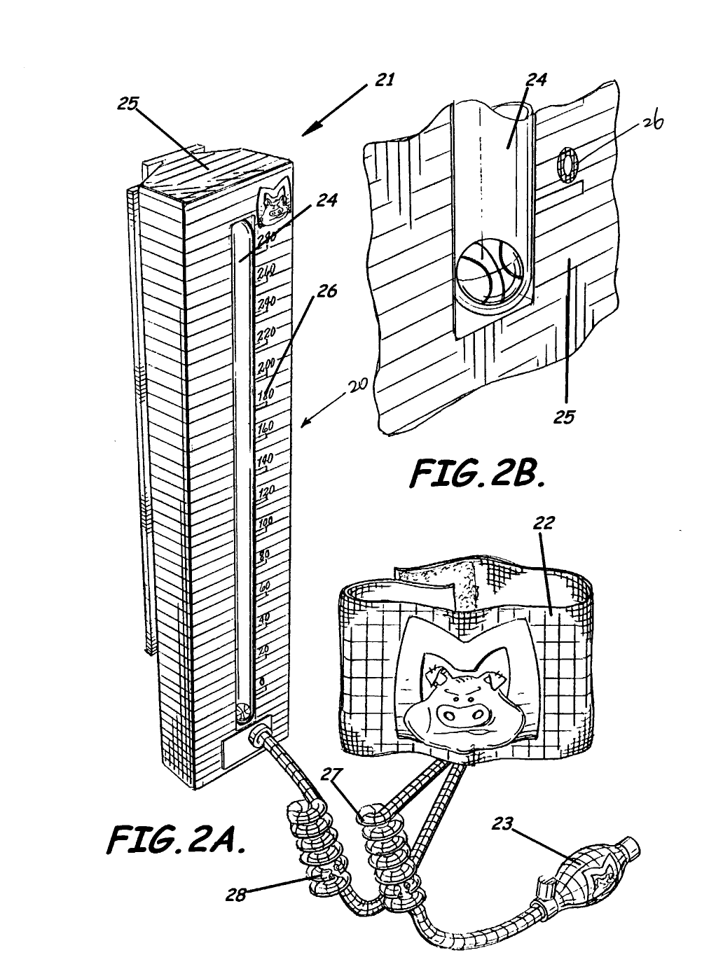

Illustrated in FIGS. 2A and 2B is a blood pressure monitor 21 for sports enthusiasts. The blood pressure monitor 21 preferably has a cuff 22 which has a fabric portion and an inflatable bladder portion and is adapted to be positioned to substantially surround a patient's arm. The blood pressure monitor 21 also preferably has a bulb 23 which is in fluid communication with the bladder portion of the cuff 22 to inflate the bladder portion in response to squeeze pressure from the user, measuring means 20 which is also in fluid communication with the bladder portion of the cuff 22 for measuring blood pressure of the patient when the cuff 22 substantially surrounds the patient's arm, and a first tube 27 having one end portion connected to and in fluid communication with the cuff 22 and the other end portion connected to and in fluid communication with the bulb 23, and a second tube 28 having one end portion connected to and in fluid communication with the cuff 22 and the other end portion connected to and in fluid communication with the measuring means 20.

The measuring means 20, for example, also can be provided by a casing 25, a tubular housing 24 which is connected to the casing 25 and has mercury material positioned therein, and measurement indicators 26 which are associated with the tubular housing 24 to indicate blood pressure measurements in response to the position of the mercury material positioned within the housing. In actual use, cuff 22 is wrapped around a patient's arm. Air is pumped into the bladder portion of the cuff 22 by means of the squeeze bulb 23. The bladder portion of the cuff 22 is then inflated to momentarily stop the flow of blood in an artery and subsequently deflated as air is released from the cuff 22. In consequence, the mercury material in the tubular housing 24 rises within the housing in response to the pressure in the cuff 22, thus enabling the measurement of the patient's blood pressure.

Each of the first tube 27 and the second tube 28 is preferably colored with at least one of a first color associated with a sports team to thereby define a first sports team color and a second contrasting color also associated with the sports team to thereby define a second sports team color. The fabric portion of the cuff 22, the surface of the bulb 23, the surface of the tubular housing 24 and/or the surface of the casing 25 are preferably colored with at least one of the first and second sports team colors. To further target the sports team, sports indicia associated with the sports team are also imprinted on the fabric portion of the cuff 22, the surface of the bulb 23, and/or the surface of the casing 25. As illustrated in FIG. 2A, the Mundo Fathogs logo is imprinted on the fabric portion of the cuff 22, the surface of the bulb 23 and the surface of the casing 25. FIG. 2B illustrates an enlarged view of a lower end portion of the tubular housing 24, which preferably has a shape substantially similar to a ball associated with the sports team, e.g. a basketball.

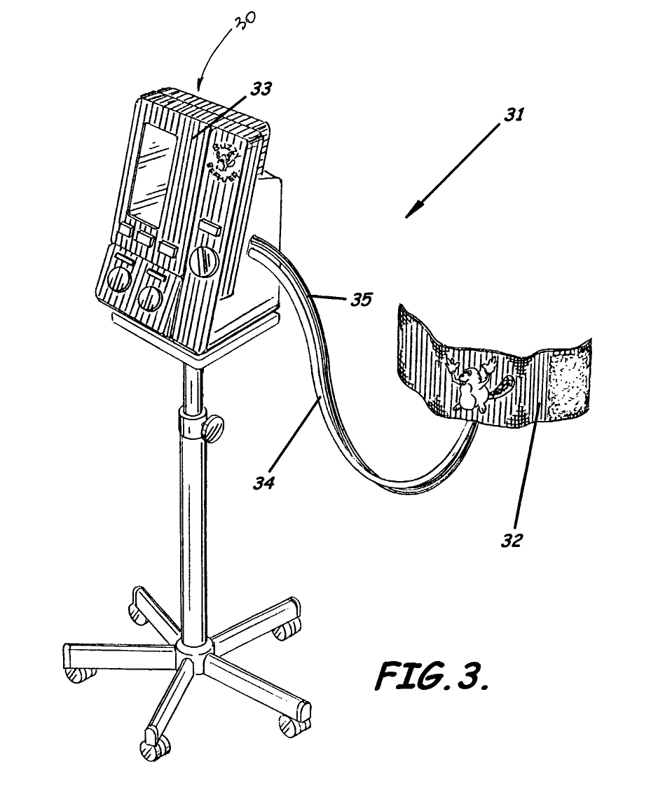

FIG. 3 illustrates another embodiment of a blood pressure monitor 31 which also is provided according to the present invention. The blood pressure monitor 31 preferably has a cuff 32 which has a fabric portion and an inflatable bladder portion and is adapted to be positioned to substantially surround a patient's arm. The monitor 31 also preferably has inflating and measuring means 30 which is adapted to be positioned to inflate and deflate the bladder portion of the cuff 32 as well as to measure the patient's blood pressure, and first tube 34 and second tube 35 each having one end portion connected to and in fluid communication with the cuff 32 and the other end portion connected to and in fluid communication with the measuring means 30.

The inflating and measuring means 30 preferably is provided by a battery-operated gauge 33. In actual use, the cuff 32 is wrapped around a patient's arm. The user presses a start button on the gauge 33 to inflate the bladder portion of the cuff 32. As air is released from the cuff 32, a reading displays on the gauge 33, which records the patient's blood pressure.

Each of the first tube 34 and second tube 35 is preferably colored with at least one of two contrasting colors associated with a sports team. The fabric portion of the cuff 32 and the surface of the gauge 33 are also preferably colored with at least one of the two contrasting sports team colors. To further target the sports team, sports indicia associated with the sports team are also imprinted on the fabric portion of the cuff 32 and the surface of the gauge 33. As illustrated in FIG. 3, the Buzzy Beaver logo is imprinted on the fabric portion of the cuff 32 and the face of the gauge 33. The sports logo is readily visible and recognizable to the user and the patient.

The present invention also advantageously provides a method of retrofitting an existing blood pressure monitor for a sports blood pressure monitor. The method preferably includes replacing first and second tubes of the existing blood pressure monitor with third and fourth tubes being visibly colored with at least one of a first color associated with a sports team to thereby define a first sports team color and a second contrasting color also associated with the sports team to thereby define a second sports team color.

The retrofitting method can further include replacing a cuff of the existing blood pressure monitor with a different cuff which is preferably colored with at least one of the first and second sports team colors. More preferably, the cuff can have sports indicia associated with the sports team imprinted thereon.

Alternatively, the retrofitting method can further include replacing a bulb of the existing blood pressure monitor with a different bulb which is preferably colored with at least one of the first and second sports team colors. More preferably, the bulb can have sports indicia associated with the sports team imprinted thereon.

The present invention further advantageously provides a method of doing business by obtaining a license from at least one sports teams to use sports team identifiers associated with the sports team in association with a medical device such as a blood pressure monitor, positioning sports team identifiers on a medical device such as a blood pressure monitor so that a potential user of the blood pressure monitor readily recognizes that the blood pressure monitor is associated with the sports team, selling the medical device, e.g., the blood pressure monitor, to the potential user, and distributing the medical device, e.g., the blood pressure monitor to the potential user after purchase of the medical device. Such method can further include advertising the medical device, e.g., the blood pressure monitor, associated with the sports team. The sports team can include at least one of the following: a school sports team, a professional sports team and a club sports team. Still preferably, the sports identifier is positioned on at least one of the following: a fabric portion of a cuff, a bulb, a surface of measuring means of the blood pressure monitor. For example, one or more licenses can be obtained and entirely new devices can be sold under the license or portions of products can be manufactured and sold for retrofit purpose, e.g., tubes, cuffs, bulbs or casings. Such devices or portions of thereof can also advantageously be sold in kits as well.

Another method of doing business according to the present invention preferably includes obtaining a license from each of a plurality of different sports teams to use sports team identifiers associated with each of the plurality of sports teams in association with a plurality of blood pressure monitors; and manufacturing a plurality of blood pressure monitors. Each of the plurality of blood pressure monitors preferably has a different sports team identifier associated therewith. Such method can further include advertising each of the plurality of blood pressure monitors associated with each of the plurality of sports teams or selling the plurality of blood pressure monitors to the potential user and distributing the plurality of blood pressure monitors to the potential user after purchase of the blood pressure monitors.

In the drawings and specification, there have been disclosed a typical preferred embodiment of the invention, and although specific terms are employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

That claimed is:

1. A blood pressure monitor for sports enthusiasts for monitoring blood pressure of a patient by a user, the blood pressure monitor comprising:
(a) a cuff adapted to be positioned to substantially surround a portion of one of the patient's arms, the cuff having a fabric portion and an inflatable bladder portion associated with the fabric portion, the fabric portion having sports indicia associated with a sports team positioned thereon and readily visible and recognizable to the user and the patient;
(b) a bulb in fluid communication with the inflatable bladder portion to inflate the bladder portion of the cuff responsive to squeeze pressure from the user;
(c) measuring means in fluid communication with the bladder portion for measuring blood pressure of the patient when the bladder portion substantially surrounds the portion of the patient's arm;
(d) a first tube having a first end portion connected to and in fluid communication with the cuff and a second end portion connected to and in fluid communication with the bulb, the first tube being visibly colored with at least one of a first color associated with the sports team to thereby define a first sports team color and a second contrasting color also associated with the sports team to thereby define a second sports team color; and
(e) a second tube having a first end portion connected to and in fluid communication with the cuff and a second end portion connected to and in fluid communication with the measuring means, the second tube being visibly colored with at least one of the first and second sports team colors.

2. The blood pressure monitor of claim 1, wherein the sports team includes at least one of the following: a school sports team, a professional sports team, and a club sports team.

3. The blood pressure monitor of claim 1, wherein the fabric portion of the cuff further is colored with at least one of the first and second sports team colors.

4. The blood pressure monitor of claim 1, wherein the bulb is colored with at least one of the first and second sports team colors.

5. The blood pressure monitor of claim 4, wherein the bulb further has the sports indicia positioned thereon and readily visible and recognizable to the user and the patient.

6. The blood pressure monitor of claim 1, wherein the first tube is colored with the first sports team color, and wherein the second tube is colored with the second sports team color.

7. The blood pressure monitor of claim 1, wherein the measuring means comprises a dial having a needle moving responsive to pressure in the cuff, and wherein face of the dial has the sports indicia positioned thereon and readily visible and recognizable to the user and the patient.

8. The blood pressure monitor of claim 1, wherein the measuring means comprises a casing including a surface thereof having the sports indicia positioned thereon and readily recognizable to the user and the patient and a tubular housing connected to the casing and having mercury material positioned therein, the mercury material being responsive to pressure in the cuff, and wherein the measuring means further comprises measurement indicators associated with the tubular housing to indicate blood pressure measurements responsive to the position of the mercury material positioned within the tubular housing.

9. The blood pressure monitor of claim 8, wherein the tubular housing further is colored with the first sports team color, and wherein the surface of the casing further is colored with the second sports team color.

10. The blood pressure monitor of claim 8, wherein a lower end portion of the tubular housing having the mercury material positioned therein further has a shape substantially similar to a ball associated with the sports team.

11. A blood pressure monitor for sports enthusiasts for monitoring blood pressure of a patient by a user, the blood pressure monitor comprising:
(a) a cuff adapted to be positioned to substantially surround a portion of one of the patient's arms, the cuff having a fabric portion and an inflatable bladder portion associated with the fabric portion;
(b) a bulb in fluid communication with the inflatable bladder portion to inflate the bladder portion of the cuff responsive to squeeze pressure from the user;
(c) measuring means in fluid communication with the bladder portion for measuring blood pressure of the patient when the bladder portion substantially surrounds the portion of the patient's arm;
(d) a first tube having a first end portion connected to and in fluid communication with the cuff and a second end portion connected to and in fluid communication with the bulb, the first tube being visibly colored with at least one of a first color associated with a sports team to thereby define a first sports team color and a second contrasting color also associated with the sports team to thereby define a second sports team color; and
(e) a second tube having a first end portion connected to and in fluid communication with the cuff and a second end portion connected to and in fluid communication with the measuring means, the second tube being visibly colored with at least one of the first and second sports team colors.

12. The blood pressure monitor of claim 11, wherein the sports team includes at least one of the following: a school sports team, a professional sports team, and a club sports team.

13. The blood pressure monitor of claim 11, wherein the fabric portion of the cuff is visibly colored with at least one of the first and second sports team color.

14. The blood pressure monitor of claim 13, wherein the fabric portion of the cuff further has sports indicia associated with the sports team positioned thereon and readily visible and recognizable to the user and the patient.

15. The blood pressure monitor of claim 11, wherein the bulb is colored with at least one of the first and second sports team colors.

16. The blood pressure monitor of claim 15, wherein the bulb further has sports indicia associated with the sports team positioned thereon and readily visible and recognizable to the user and the patient.

17. The blood pressure monitor of claim 11, wherein the first tube is colored with the first sports team color, and wherein the second tube is colored with the second sports team color.

18. The blood pressure monitor of claim 11, wherein the measuring means comprises a dial having a needle moving responsive to pressure in the cuff, and wherein face of the dial has sports indicia associated with the sports team positioned thereon and readily visible and recognizable to the user and the patient.

19. The blood pressure monitor of claim 11, wherein the measuring means comprises a casing including a surface thereof having sports indicia associated with the sports team positioned thereon and readily recognizable to the user and the patient and a tubular housing connected to the casing and having mercury material positioned therein, the mercury material being responsive to pressure in the cuff, and wherein the measuring means further comprises measurement indicators associated with the tubular housing to indicate blood pressure measurements responsive to the position of the mercury material positioned within the tubular housing.

20. The blood pressure monitor of claim 19, wherein the tubular housing further is colored with the first sports team color, and wherein the surface of the casing further is colored with the second sports team color.

21. The blood pressure monitor of claim 19, wherein a lower end portion of the tubular housing having the mercury material positioned therein further has a shape substantially similar to a ball associated with the sports team.

22. A blood pressure monitor for sports enthusiasts for monitoring blood pressure of a patient by a user, the blood pressure monitor comprising:
(a) a cuff adapted to be positioned to substantially surround a portion of one of the patient's arms, the cuff having a fabric portion and an inflatable bladder portion associated with the fabric portion, the fabric portion having sports indicia associated with a sports team positioned thereon and readily visible and recognizable to the user and the patient;
(b) a gauge in fluid communication with the bladder portion of the cuff to inflate and deflate the bladder portion responsive to the user and to measure blood pressure of the patient when the bladder portion substantially surrounds the portion of the patient's arm; and
(c) first and second tubes each having a first end portion connected to and in fluid communication with the cuff and a second end portion connected to and in fluid communication with the gauge, the first and second tubes being visibly colored with at least one of a first color associated with the sports team to thereby define a first sports team color and a second contrasting color also associated with the sports team to thereby define a second sports team color.

23. The blood pressure monitor of claim 22, wherein the sports team includes at least one of the following: a school sports team, a professional sports team, and a club sports team.

24. The blood pressure monitor of claim 22, wherein the fabric portion of the cuff further is colored with at least one of the first and second sports team colors.

25. The blood pressure monitor of claim 22, wherein the gauge has a surface visibly colored with at least one of the first and second sports team colors.

26. The blood pressure monitor of claim 25, wherein the gauge surface further has the sports indicia positioned thereon and readily visible and recognizable to the user and the patient.

27. The blood pressure monitor of claim 22, wherein the first tube is colored with the first sports team color, and wherein the second tube is colored with the second sports team color.

28. A blood pressure monitor for sports enthusiasts for monitoring blood pressure of a patient by a user, the blood pressure monitor comprising:
(a) a cuff adapted to be positioned to substantially surround a portion of one of the patient's arms, the cuff having a fabric portion and an inflatable bladder portion associated with the fabric portion;
(b) a gauge in fluid communication with the bladder portion of the cuff to inflate and deflate the bladder portion responsive to the user and to measure blood pressure of the patient when the bladder portion substantially surrounds the portion of the patient's arm; and
(c) first and second tubes each having a first end portion connected to and in fluid communication with the cuff and a second end portion connected to and in fluid communication with the gauge, the first and second tubes being visibly colored with at least one of a first color associated with the sports team to thereby define a first sports team color and a second contrasting color also associated with the sports team to thereby define a second sports team color.

29. The blood pressure monitor of claim 28, wherein the sports team includes at least one of the following: a school sports team, a professional sports team, and a club sports team.

30. The blood pressure monitor of claim 28, wherein the fabric portion of the cuff is visibly colored with at least one of the first and second sports team colors.

31. The blood pressure monitor of claim 30, wherein the fabric portion of the cuff further has sports indicia associated with the sports team positioned thereon and readily visible and recognizable to the user and the patient.

32. The blood pressure monitor of claim 28, wherein the gauge has a surface visibly colored with at least one of the first and second sports team colors.

33. The blood pressure monitor of claim 32, wherein the gauge surface further has sports indicia associated with the sports team positioned thereon and readily visible and recognizable to the user and the patient.

34. The blood pressure monitor of claim 28, wherein the first tube is colored with the first sports team color, and wherein the second tube is colored with the second sports team color.

35. A method of retrofitting an existing blood pressure monitor for sports enthusiasts, the method comprising:
replacing first and second tubes of the existing blood pressure monitor with third and fourth tubes, each of the third and fourth tubes being visibly colored with at least one of a first color associated with a sports team to thereby define a first sports team color and a second contrasting color also associated with the sports team to thereby define a second sports team color.

36. The method of claim 35, further comprising:
replacing a first cuff of the existing blood pressure monitor with a second cuff being visibly colored with at least one of the first and second sports team colors.

37. The method of claim 36, further comprising:
positioning sports indicia on the second cuff, the sports indicia having colors corresponding to the first and second sports team colors associated with the third and fourth tubes.

38. The method of claim 35, further comprising:
replacing a first bulb of the existing blood pressure monitor with a second bulb being visibly colored with at least one of the first and second sports team colors.

39. The method of claim 38, further comprising:
positioning sports indicia on the second bulb, the sports indicia having colors corresponding to the first and second sports team colors associated with the third and fourth tubes.

40. A method of doing business, the method comprising:
obtaining a license from at least one sports teams to use sports team identifiers associated with the sports team in association with a blood pressure monitor;
positioning sports team identifiers on a blood pressure monitor so that a potential user of the blood pressure monitor readily recognizes that the blood pressure monitor is associated with the sports team;
selling the blood pressure monitor to the potential user; and
distributing the blood pressure monitor to the potential user after purchase of the blood pressure monitor.

41. The method of claim 35, further comprising:
advertising the blood pressure monitor associated with the sports team.

42. The method of claim 35, wherein the sports team includes at least one of the following: a school sports team, a professional sports team, and a club sports team.

43. The method of claim 35, wherein the sports identifier is positioned on at least one of the following: a fabric portion of a cuff, a bulb, a surface of measuring means of the blood pressure monitor.

44. A method of doing business, the method comprising:
obtaining a license from each of a plurality of different sports teams to use sports team identifiers associated with each of the plurality of sports teams in association with a plurality of blood pressure monitors; and
manufacturing a plurality of blood pressure monitors, each blood pressure monitor having a different sports team identifier associated therewith.

45. The method of claim 39, further comprising:
advertising each of the plurality of blood pressure monitors associated with each of the plurality of sports teams.

46. The method of claim 39, further comprising:
selling the plurality of blood pressure monitors to the potential user; and
distributing the plurality of blood pressure monitors to the potential user after purchase of the blood pressure monitors.

47. The method of claim 39, wherein the sports team includes at least one of the following: a school sports team, a professional sports team, and a club sports team.

48. The method of claim 39, wherein the sports identifier is positioned on at least one of the following: a fabric portion of a cuff, a bulb, a surface of measuring means of the blood pressure monitor.
